(19) United States
(12) Patent Application Publication
(10) Pub. No.: US 2015/0141351 A1
(43) Pub. Date: May 21, 2015

(54) Solid Pharmaceutical Compositions

(71) Applicant: AbbVie Deutschland GmbH & Co. KG, Wiesbaden (DE)

(72) Inventors: Faith Durak, Wiesbaden (DE); Peter Hoeling, Waechtersbach (DE); Thomas Kessler, Schifferstadt (DE); Drazen Kostelac, Mannheim (DE); Bernd Liepold, Dossenheim (DE); Anna Moosmann, Winterbach (DE); Mirko Pauli, Ludwigshafen (DE); Karin Rosenblatt, Mannheim (DE)

(73) Assignee: ABBVIE DEUTSCHLAND GMBH & CO. KG, Wiesbaden (DE)

(21) Appl. No.: 14/543,135

(22) Filed: Nov. 17, 2014

Related U.S. Application Data
(60) Provisional application No. 61/905,537, filed on Nov. 18, 2013.
(60) Provisional application No. 61/911,784, filed on Dec. 4, 2013.

(51) Int. Cl. A61K 38/06; A61K 31/427; A61K 9/20; A61K 38/05

(52) U.S. Cl. CPC A61K 38/06; A61K 38/05; A61K 31/427; A61K 9/2027; A61K 9/2013
USPC 514/21.9

(57) ABSTRACT

The present invention features solid pharmaceutical compositions comprising Compound 1 (or a pharmaceutically acceptable salt thereof), Compound 2 (or a pharmaceutically acceptable salt thereof), and ritonavir (a pharmaceutically acceptable salt thereof), which are co-formulated in amorphous solid dispersion comprising a pharmaceutically acceptable hydrophilic polymer and a pharmaceutically acceptable surfactant.

### FIELD OF THE INVENTION

The present invention relates to solid pharmaceutical compositions comprising anti-HCV compounds and methods of using the same to treat HCV infection.

### BACKGROUND

The hepatitis C virus (HCV) is an RNA virus belonging to the Hepacivirus genus in the Flaviviridae family. The enveloped HCV virion contains a positive stranded RNA genome encoding all known virus-specific proteins in a single, uninterrupted, open reading frame. The open reading frame comprises approximately 9500 nucleotides and encodes a single large polyprotein of about 3000 amino acids. The polyprotein comprises a core protein, envelope proteins E1 and E2, a membrane bound protein p7, and the non-structural proteins NS2, NS3, NS4A, NS4B, NS5A and NS5B.

HCV infection is associated with progressive liver pathology, including cirrhosis and hepatocellular carcinoma. Chronic hepatitis C may be treated with peginterferon-alpha in combination with ribavirin. Substantial limitations to efficacy and tolerability remain as many users suffer from side effects, and viral elimination from the body is often inadequate. Therefore, there is a need for new drugs to treat HCV infection.

### DETAILED DESCRIPTION

In one aspect, the present invention features solid pharmaceutical compositions useful for treating HCV. The solid pharmaceutical composition comprises:

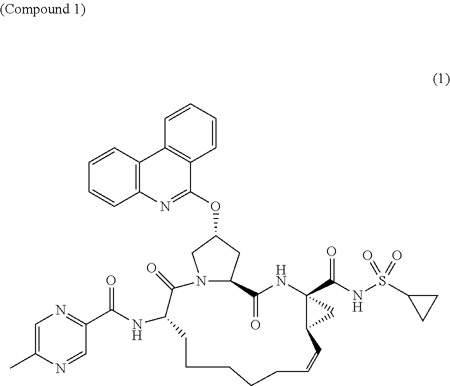 [SMILES: Cc1cnc(C(=O)N[C@H]2CCCCC/C=C\[C@@H]3C[C@@]3(C(=O)NS(=O)(=O)C3CC3)NC(=O)[C@@H]3C[C@@H](Oc4nc5ccccc5c5ccccc45)CN3C2=O)cn1]

or a pharmaceutically acceptable salt thereof,

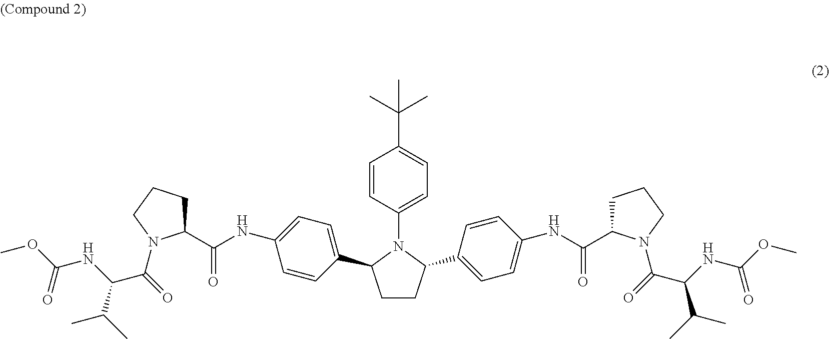

or a pharmaceutically acceptable salt thereof,

(3) ritonavir or a pharmaceutically acceptable salt thereof,

(4) a pharmaceutically acceptable hydrophilic polymer, and

(5) a pharmaceutically acceptable surfactant,

all of which are co-formulated in amorphous solid dispersion. Compound 1 is also known as (2R,6S,13aS,14aR,16aS,Z)-N-(cyclopropylsulfonyl)-6-(5-methylpyrazine-2-carboxamido)-5,16-dioxo-2-(phenanthridin-6-yloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxamide, which is a potent HCV protease inhibitor. The synthesis and formulation of Compound 1 are described in U.S. Patent Application Publication Nos. 2010/0144608 and 2011/0312973. Compound 2 is also known as dimethyl (2S,2′S)-1,1′-((2S,2′S)-2,2′-(4,4′-((2S,5S)-1-(4-tert-butylphenyl)pyrrolidine-2,5-diyl)bis(4,1-phenylene))bis(azanediyl)bis(oxomethylene)bis(pyrrolidine-2,1-diyl))bis(3-methyl-1-oxobutane-2,1-diyl)dicarbamate, which is a potent HCV NS5A inhibitor. The synthesis and formulation of Compound 2 are described in U.S. Patent Application Publication Nos. 2010/0317568 and 2012/0258909.

Compound 1 and Compound 2 are both poorly soluble compounds, and have been each separately formulated in amorphous solid dispersions. These solid dispersions can then be mixed together to provide a pharmaceutical dosage form that contains both Compound 1 and Compound 2.

Drug loads in conventional amorphous solid dispersions of poorly soluble compounds are preferably no more than 15% by weight. This is because higher drugs loads can lead to substantial reduction in drug release. As a result, when separate solid dispersions of Compound 1 and Compound 2, each having no more than 15% drug load, are mixed and compressed, the resulting pharmaceutical dosage form requires a large size to accommodate the relatively low drug load.

Moreover, hot melt extrusion, the preferred method to prepare amorphous solid dispersion, often involves the use of high temperature to assist the formation of a melt that is composed of all components of the final solid dispersion. Certain drug substances, such as ritonavir, can reach unacceptable degradation levels at temperatures of beyond 140° C., which considerably limits the use of the hot melt extrusion process to co-formulate ritonavir with other drug substance(s) when the other drug substance(s) requires higher temperatures to form a suitable melt.

It was surprisingly found that when Compound 1, Compound 2 and ritonavir were co-formulated in amorphous solid dispersion, the total drug load in the amorphous solid dispersion can be significantly increased without compromising drug release. This allows the preparation of smaller solid dosage forms (e.g., tablets) that contain all three drug substances. It was also unexpected discovered that when Compound 1, Compound 2 and ritonavir were co-extruded in the hot melt extrusion process, ritonavir became less susceptible to high temperatures, and even at a temperature of 165° C., ritonavir degradation was well within the acceptable levels.

Preferably, in any aspect, embodiment, example, preference and composition of the invention, the total weight of Compound 1, Compound 2 and ritonavir in the amorphous solid dispersion ranges from 20% to 40% by weight relative to the total weight of the amorphous solid dispersion. More preferably, in any aspect, embodiment, example, preference and composition of the invention, the total weight of Compound 1, Compound 2 and ritonavir in the amorphous solid dispersion ranges from 20% to 30% by weight relative to the total weight of the amorphous solid dispersion. Highly preferably, in any aspect, embodiment, example, preference and composition of the invention, the total weight of Compound 1, Compound 2 and ritonavir in the amorphous solid dispersion ranges from 25% to 30% by weight relative to the total weight of the amorphous solid dispersion.

In any aspect, embodiment, example, preference and composition of the invention, Compound 1 in the amorphous solid dispersion can range from 10% to 20% by weight relative to the total weight of the amorphous solid dispersion; Compound 2 in the amorphous solid dispersion can range from 2% to 5% by weight relative to the total weight of the amorphous solid dispersion; and ritonavir in the amorphous solid dispersion can range from 5% to 15% by weight relative to the total weight of the amorphous solid dispersion.

In any aspect, embodiment, example, preference and composition of the invention, Compound 1 in the amorphous solid dispersion can range from 15% to 20% by weight relative to the total weight of the amorphous solid dispersion; Compound 2 in the amorphous solid dispersion can range from 2% to 3% by weight relative to the total weight of the amorphous solid dispersion; and ritonavir in the amorphous solid dispersion can range from 10% to 15% by weight relative to the total weight of the amorphous solid dispersion.

In any aspect, embodiment, example, preference and composition of the invention, Compound 1 in the amorphous solid dispersion can be of about 15% by weight relative to the total weight of the amorphous solid dispersion; Compound 2 in the amorphous solid dispersion can range from 2% to 3% by weight relative to the total weight of the amorphous solid dispersion; and ritonavir in the amorphous solid dispersion can be of about 10% by weight relative to the total weight of the amorphous solid dispersion.

In any aspect, embodiment, example, preference and composition of the invention, the amount of Compound 1 in the amorphous solid dispersion can be, for example, 75 mg; the amount of Compound 2 in the amorphous solid dispersion can be, for example, 12.5 mg; and the amount of ritonavir in the amorphous solid dispersion can be, for example, 50 mg.

In any aspect, embodiment, example, preference and composition of the invention, the amorphous solid dispersion can comprise from 50% to 75% by weight, relative to the total weight of the amorphous solid dispersion, of said polymer, and from 2% to 15% by weight, relative to the total weight of the amorphous solid dispersion, of said surfactant.

In any aspect, embodiment, example, preference and composition of the invention, the amorphous solid dispersion can comprise from 50% to 70% by weight, relative to the total weight of the amorphous solid dispersion, of said polymer, and from 5% to 15% by weight, relative to the total weight of the amorphous solid dispersion, of said surfactant.

In any aspect, embodiment, example, preference and composition of the invention, the amorphous solid dispersion can comprise from 55% to 65% by weight, relative to the total weight of the amorphous solid dispersion, of said polymer, and from 5% to 10% by weight, relative to the total weight of the amorphous solid dispersion, of said surfactant.

In any aspect, embodiment, example, preference and composition of the invention, the amorphous solid dispersion can comprise from 60% to 65% by weight, relative to the total weight of the amorphous solid dispersion, of said polymer, and from 5% to 10% by weight, relative to the total weight of the amorphous solid dispersion, of said surfactant.

In any aspect, embodiment, example, preference and composition of the invention, the amorphous solid dispersion can be prepared as a compressed core (e.g., a tablet core), onto which another layer of other excipients or ingredients can be optionally added. For example, the amorphous solid dispersion can be milled and then mixed with other excipients or ingredients, and the mixture can be compressed to form the core.

In one embodiment, said polymer in the amorphous solid dispersion can range from 50% to 75% by weight relative to the total weight of said compressed core, and said surfactant in the amorphous solid dispersion can range from 5 to 15% by weight relative to the total weight of said compressed core.

In another embodiment, said polymer in the amorphous solid dispersion can range from 50% to 70% by weight relative to the total weight of said compressed core, and said surfactant in the amorphous solid dispersion can range from 5 to 15% by weight relative to the total weight of said compressed core.

In yet another embodiment, said polymer in the amorphous solid dispersion can range from 55% to 65% by weight relative to the total weight of said compressed core, and said surfactant in the amorphous solid dispersion can range from 5 to 10% by weight relative to the total weight of said compressed core.

In yet another embodiment, said polymer in the amorphous solid dispersion can range from 60% to 65% by weight relative to the total weight of said compressed core, and said surfactant in the amorphous solid dispersion can range from 5 to 10% by weight relative to the total weight of said compressed core.

In any aspect, embodiment, example, preference and composition of the invention, the total weight of Compound 1, Compound 2 and ritonavir in the amorphous solid dispersion can range from 20% to 40% by weight relative to the total weight of the compressed core.

In any aspect, embodiment, example, preference and composition of the invention, the total weight of Compound 1, Compound 2 and ritonavir in the amorphous solid dispersion can range from 20% to 30% by weight relative to the total weight of the compressed core.

In any aspect, embodiment, example, preference and composition of the invention, the total weight of Compound 1, Compound 2 and ritonavir in the amorphous solid dispersion can range from 25% to 30% by weight relative to the total weight of the compressed core.

In any aspect, embodiment, example, preference and composition of the invention, Compound 1 in the amorphous solid dispersion can range from 10% to 20% by weight relative to the total weight of the compressed core; Compound 2 in the amorphous solid dispersion can range from 2% to 5% by weight relative to the total weight of the compressed core; and ritonavir in the amorphous solid dispersion can range from 5% to 15% by weight relative to the total weight of the compressed core.

In any aspect, embodiment, example, preference and composition of the invention, Compound 1 in the amorphous solid dispersion can range from 15% to 20% by weight relative to the total weight of the compressed core; Compound 2 in the amorphous solid dispersion can range from 2% to 3% by weight relative to the total weight of the compressed core; and ritonavir in the amorphous solid dispersion can range from 10% to 15% by weight relative to the total weight of the compressed core.

In any aspect, embodiment, example, preference and composition of the invention, Compound 1 in the amorphous solid dispersion can be of about 15% by weight relative to the total weight of the compressed core; Compound 2 in the amorphous solid dispersion can range from 2% to 3% by weight relative to the total weight of the compressed core; and ritonavir in the amorphous solid dispersion can be of about 10% by weight relative to the total weight of the compressed core.

In any aspect, embodiment, example, preference and composition of the invention, the hydrophilic polymer can have a T_{g} of at least 50° C.; preferably, the hydrophilic polymer has a T_{g} of at least 80° C.; more preferably, the hydrophilic polymer has a T_{g} of at least 100° C. For example, the hydrophilic polymer can have a T_{g} of from 80° C. to 180° C., or from 100° C. to 150° C.

Preferably, the hydrophilic polymer employed in the present invention is water-soluble. A solid pharmaceutical composition of the invention can also comprise poorly water-soluble or water-insoluble polymers, such as cross-linked polymers. The hydrophilic polymer comprised in a solid pharmaceutical composition of the invention preferably has an apparent viscosity, when dissolved at 20° C. in an aqueous solution at 2% (w/v), of 1 to 5000 mPa·s, and more preferably of 1 to 700 mPa·s, and most preferably of 5 to 100 mPa·s.

In any aspect, embodiment, example and composition of the invention, the hydrophilic polymer can be selected from homopolymer of N-vinyl lactam, copolymer of N-vinyl lactam, cellulose ester, cellulose ether, polyalkylene oxide, polyacrylate, polymethacrylate, polyacrylamide, polyvinyl alcohol, vinyl acetate polymer, oligosaccharide, polysaccharide, or combinations thereof. Non-limiting examples of suitable hydrophilic polymers include homopolymer of N-vinyl pyrrolidone, copolymer of N-vinyl pyrrolidone, copolymer of N-vinyl pyrrolidone and vinyl acetate, copolymer of N-vinyl pyrrolidone and vinyl propionate, polyvinylpyrrolidone, methylcellulose, ethylcellulose, hydroxyalkylcelluloses, hydroxypropylcellulose, hydroxyalkylalkylcellulose, hydroxypropylmethylcellulose, cellulose phthalate, cellulose succinate, cellulose acetate phthalate, hydroxypropylmethylcellulose phthalate, hydroxypropylmethylcellulose succinate, hydroxypropylmethylcellulose acetate succinate, polyethylene oxide, polypropylene oxide, copolymer of ethylene oxide and propylene oxide, methacrylic acid/ethyl acrylate copolymer, methacrylic acid/methyl methacrylate copolymer, butyl methacrylate/2-dimethylaminoethyl methacrylate copolymer, poly(hydroxyalkyl acrylate), poly(hydroxyalkyl methacrylate), copolymer of vinyl acetate and crotonic acid, partially hydrolyzed polyvinyl acetate, carrageenan, galactomannan, xanthan gum, or combinations thereof.

In any aspect, embodiment, example, preference and composition of the invention, the polymer preferably is copovidone.

In any aspect, embodiment, example, preference and composition of the invention, the surfactant can have an HLB value of at least 10. Surfactants having an HLB value of less than 10 can also be used. Preferably, in any aspect, embodiment and example of the invention, a solid pharmaceutical composition comprises a surfactant having an HLB of at least 10 and another surfactant having an HLB value of less than 10, and both surfactants are co-formulated in the amorphous solid dispersion.

In any aspect, embodiment, example, preference and composition of the invention, the surfactant can be selected from polyoxyethylene castor oil derivates, mono fatty acid ester of polyoxyethylene sorbitan, polyoxyethylene alkyl ether, polyoxyethylene alkylaryl ether, polyethylene glycol fatty acid ester, alkylene glycol fatty acid mono ester, sucrose fatty acid ester, sorbitan fatty acid mono ester, or combinations thereof. Non-limiting examples of suitable surfactants include polyoxyethyleneglycerol triricinoleate or polyoxyl 35 castor oil (Cremophor® EL; BASF Corp.) or polyoxyethyleneglycerol oxystearate such as polyethylenglycol 40 hydrogenated castor oil (Cremophor® RH 40, also known as polyoxyl 40 hydrogenated castor oil or macrogolglycerol hydroxystearate) or polyethylenglycol 60 hydrogenated castor oil (Cremophor® RH 60), mono fatty acid ester of polyoxyethylene sorbitan, such as mono fatty acid ester of polyoxyethylene (20) sorbitan, e.g. polyoxyethylene (20) sorbitan monooleate (Tween® 80), polyoxyethylene (20) sorbitan monostearate (Tween® 60), polyoxyethylene (20) sorbitan monopalmitate (Tween® 40) or polyoxyethylene (20) sorbitan monolaurate (Tween® 20), polyoxyethylene (3) lauryl ether, polyoxyethylene (5) cetyl ether, polyoxyethylene (2) stearyl ether, polyoxyethylene (5) stearyl ether, polyoxyethylene (2) nonylphenyl ether, polyoxyethylene (3) nonylphenyl ether, polyoxyethylene (4) nonylphenyl ether, polyoxyethylene (3) octylphenyl ether, PEG-200 monolaurate, PEG-200 dilaurate, PEG-300 dilaurate, PEG-400 dilaurate, PEG-300 distearate, PEG-300 dioleate, propylene glycol monolaurate (e.g., Lauroglycol), sucrose monostearate, sucrose distearate, sucrose monolaurate, sucrose dilaurate, sorbitan mono laurate, sorbitan monooleate, sorbitan monopalnitate, sorbitan stearate, or combinations thereof.

In any aspect, embodiment, example, preference and composition of the invention, the surfactant preferably is D-alpha-tocopheryl polyethylene glycol 1000 succinate (vitamin E TPGS).

In any aspect, embodiment, example, preference and composition of the invention, the polymer preferably is copovidone, and the surfactant preferably is vitamin E TPGS.

More preferably, in any aspect, embodiment, example, preference and composition of the invention, the polymer is copovidone, the surfactant is vitamin E TPGS, and the amorphous solid dispersion further comprises propylene glycol monolaurate (e.g., Lauroglycol). Propylene glycol monolaurate can range, for example, from 1% to 5% by weight relative to the total weight of said amorphous solid dispersion. Propylene glycol monolaurate can also range, for example, from 1% to 3% by weight relative to the total weight of said amorphous solid dispersion.

In one embodiment, the solid pharmaceutical composition of the invention comprises 75 mg Compound 1, 12.5 mg Compound 2, and 50 mg ritonavir, all of which are co-formulated with a pharmaceutically acceptable hydrophilic polymer and a pharmaceutically acceptable surfactant in amorphous solid dispersion, wherein the polymer in the amorphous solid dispersion ranges from 50% to 70% by weight relative to the total weight of said amorphous solid dispersion, and the surfactant in the amorphous solid dispersion ranges from 5 to 15% by weight relative to the total weight of said amorphous solid dispersion. The polymer preferably is copovidone, and the surfactant preferably is vitamin E TPGS; and the amorphous solid dispersion preferably further comprises from 1% to 5% by weight (more preferably, from 1 to 3% by weight) of propylene glycol monolaurate, relative to the total weight of said amorphous solid dispersion.

In another embodiment, the solid pharmaceutical composition of the invention comprises 75 mg Compound 1, 12.5 mg Compound 2, and 50 mg ritonavir, all of which are co-formulated with a pharmaceutically acceptable hydrophilic polymer and a pharmaceutically acceptable surfactant in amorphous solid dispersion, wherein the polymer in the amorphous solid dispersion ranges from 55% to 65% by weight relative to the total weight of said amorphous solid dispersion, and the surfactant in the amorphous solid dispersion ranges from 5 to 10% by weight relative to the total weight of said amorphous solid dispersion. The polymer preferably is copovidone, and the surfactant preferably is vitamin E TPGS; and the amorphous solid dispersion preferably further comprises from 1% to 5% by weight (more preferably, from 1 to 3% by weight) of propylene glycol monolaurate, relative to the total weight of said amorphous solid dispersion.

In yet another embodiment, the solid pharmaceutical composition of the invention comprises 75 mg Compound 1, 12.5 mg Compound 2, and 50 mg ritonavir, all of which are co-formulated with a pharmaceutically acceptable hydrophilic polymer and a pharmaceutically acceptable surfactant in amorphous solid dispersion, wherein the polymer in the amorphous solid dispersion ranges from 60% to 65% by weight relative to the total weight of said amorphous solid dispersion, and the surfactant in the amorphous solid dispersion ranges from 5 to 10% by weight relative to the total weight of said amorphous solid dispersion. The polymer preferably is copovidone, and the surfactant preferably is vitamin E TPGS; and the amorphous solid dispersion preferably further comprises from 1% to 5% by weight (more preferably, from 1 to 3% by weight) of propylene glycol monolaurate, relative to the total weight of said amorphous solid dispersion.

In yet another embodiment, the solid pharmaceutical composition comprises a compressed core which includes 75 mg Compound 1, 12.5 mg Compound 2, and 50 mg ritonavir, all of which are co-formulated with a pharmaceutically acceptable hydrophilic polymer and a pharmaceutically acceptable surfactant in amorphous solid dispersion, wherein the total weight of the compressed core is no more than 800 mg. The polymer preferably is copovidone, and the surfactant preferably is vitamin E TPGS; and the amorphous solid dispersion preferably further comprises propylene glycol monolaurate.

In yet another embodiment, the solid pharmaceutical composition comprises a compressed core which includes 75 mg Compound 1, 12.5 mg Compound 2, and 50 mg ritonavir, all of which are co-formulated with a pharmaceutically acceptable hydrophilic polymer and a pharmaceutically acceptable surfactant in amorphous solid dispersion, wherein the total weight of the compressed core is no more than 700 mg. The polymer preferably is copovidone, and the surfactant preferably is vitamin E TPGS; and the amorphous solid dispersion preferably further comprises propylene glycol monolaurate.

In yet another embodiment, the solid pharmaceutical composition comprises a compressed core which includes 75 mg Compound 1, 12.5 mg Compound 2, and 50 mg ritonavir, all of which are co-formulated with a pharmaceutically acceptable hydrophilic polymer and a pharmaceutically acceptable surfactant in amorphous solid dispersion, wherein the total weight of the compressed core is no more than 600 mg. The polymer preferably is copovidone, and the surfactant preferably is vitamin E TPGS; and the amorphous solid dispersion preferably further comprises propylene glycol monolaurate.

In yet another embodiment, the solid pharmaceutical composition comprises a compressed core which includes 75 mg Compound 1, 12.5 mg Compound 2, and 50 mg ritonavir, all of which are co-formulated with a pharmaceutically acceptable hydrophilic polymer and a pharmaceutically acceptable surfactant in amorphous solid dispersion, wherein the total weight of the compressed core is no more than 500 mg. The polymer preferably is copovidone, and the surfactant preferably is vitamin E TPGS; and the amorphous solid dispersion preferably further comprises propylene glycol monolaurate.

In yet another embodiment, the solid pharmaceutical composition comprises a compressed core which includes 75 mg Compound 1, 12.5 mg Compound 2, and 50 mg ritonavir, all of which are co-formulated with a pharmaceutically acceptable hydrophilic polymer and a pharmaceutically acceptable surfactant in amorphous solid dispersion, wherein the total weight of the compressed core ranges from 400 mg to 500 mg. The polymer preferably is copovidone, and the surfactant preferably is vitamin E TPGS; and the amorphous solid dispersion preferably further comprises propylene glycol monolaurate.

In yet another embodiment, the solid pharmaceutical composition comprises a compressed core which includes 75 mg Compound 1, 12.5 mg Compound 2, and 50 mg ritonavir, all of which are co-formulated with a pharmaceutically acceptable hydrophilic polymer and a pharmaceutically acceptable surfactant in amorphous solid dispersion, wherein the total weight of the compressed core ranges from 500 mg to 600 mg. The polymer preferably is copovidone, and the surfactant preferably is vitamin E TPGS; and the amorphous solid dispersion preferably further comprises propylene glycol monolaurate.

In yet another embodiment, the solid pharmaceutical composition comprises a compressed core which includes 75 mg Compound 1, 12.5 mg Compound 2, and 50 mg ritonavir, all of which are co-formulated with a pharmaceutically acceptable hydrophilic polymer and a pharmaceutically acceptable surfactant in amorphous solid dispersion, wherein the total weight of the compressed core ranges from 600 mg to 700 mg. The polymer preferably is copovidone, and the surfactant preferably is vitamin E TPGS; and the amorphous solid dispersion preferably further comprises propylene glycol monolaurate.

In yet another embodiment, the solid pharmaceutical composition comprises a compressed core which includes 75 mg Compound 1, 12.5 mg Compound 2, and 50 mg ritonavir, all of which are co-formulated with a pharmaceutically acceptable hydrophilic polymer and a pharmaceutically acceptable surfactant in amorphous solid dispersion, wherein the total weight of the compressed core ranges from 450 mg to 500 mg. The polymer preferably is copovidone, and the surfactant preferably is vitamin E TPGS; and the amorphous solid dispersion preferably further comprises propylene glycol monolaurate.

In yet another embodiment, the solid pharmaceutical composition comprises a compressed core which includes 75 mg Compound 1, 12.5 mg Compound 2, and 50 mg ritonavir, all of which are co-formulated with a pharmaceutically acceptable hydrophilic polymer and a pharmaceutically acceptable surfactant in amorphous solid dispersion, wherein the total weight of the compressed core is about 500 mg. The polymer preferably is copovidone, and the surfactant preferably is vitamin E TPGS; and the amorphous solid dispersion preferably further comprises propylene glycol monolaurate.

In any aspect, embodiment, example, preference and composition of the invention, the amorphous solid dispersion preferably is solid solution.

In any aspect, embodiment, example, preference and composition of the invention, the amorphous solid dispersion preferably is glassy solution.

In any aspect, embodiment, example, preference and composition of the invention, the amorphous solid dispersion preferably comprises or consists of a single-phase (defined in thermodynamics) in which Compound 1, Compound 2 and ritonavir are molecularly dispersed in a matrix containing the pharmaceutically acceptable hydrophilic polymer and the pharmaceutically acceptable surfactant. Thermal analysis of the amorphous solid dispersion using differential scanning calorimetry (DSC) typically shows only one single T_{g}, and the amorphous solid dispersion typically does not contain any detectable crystalline compound as measured by X-ray powder diffraction spectroscopy.

In any aspect, embodiment, example, preference and composition of the invention, the solid pharmaceutical composition of the invention can be a tablet.

In any aspect, embodiment, example, preference and composition of the invention, the solid pharmaceutical composition of the invention can be prepared into other suitable dosage forms, such as capsule, dragee, granule, or powder.

A solid pharmaceutical composition of the invention can further comprise another anti-HCV agent, for example, an agent selected from HCV helicase inhibitors, HCV polymerase inhibitors, HCV protease inhibitors, HCV NS5A inhibitors, CD81 inhibitors, cyclophilin inhibitors, or internal ribosome entry site (IRES) inhibitors.

In another aspect, the present invention features processes of making a solid pharmaceutical composition of the invention. The processes comprise (1) preparing a melt comprising Compound 1 (or a pharmaceutically acceptable salt thereof), Compound 2 (or a pharmaceutically acceptable salt thereof), ritonavir (or a pharmaceutically acceptable salt thereof), a pharmaceutically acceptable hydrophilic polymer, and a pharmaceutically acceptable surfactant; and (2) solidifying said melt. The solidified melt can comprise any amorphous solid dispersion described or contemplated herein. The processes can further comprise milling the solidified melt, followed by compressing the milled product with one or more other excipients or ingredients to form a tablet core. These other excipients or ingredients can include, for example, coloring agents, flavoring agents, lubricants or preservatives.

In one embodiment, the melt is formed at a temperature of from 150 to 180° C. In another embodiment, the melt is formed at a temperature of from 150 to 170° C. In yet another embodiment, the melt is formed at a temperature of from 150 to 160° C. In yet another embodiment, the melt is formed at a temperature of from 160 to 170° C.

Any amorphous solid dispersion described or contemplated herein, including any amorphous solid dispersion described or contemplated in any aspect, embodiment, example, preference and composition of the invention, can be prepared according to any process described or contemplated herein.

In still another aspect, the present invention features solid pharmaceutical compositions prepared according to a process of the invention. Any process described or contemplated herein can be used to prepare a solid pharmaceutical composition comprising Compound 1 (or a pharmaceutically acceptable salt thereof), Compound 2 (or a pharmaceutically acceptable salt thereof), ritonavir (or a pharmaceutically acceptable salt thereof), a pharmaceutically acceptable hydrophilic polymer, and a pharmaceutically acceptable surfactant.

The present invention further features methods of using a solid pharmaceutical composition of the invention to treat HCV infection. The methods comprise administering a solid pharmaceutical composition of the invention to a patient in need thereof.

The amorphous solid dispersion employed in the present invention can be prepared by a variety of techniques such as, without limitation, melt-extrusion, spray-drying, co-precipitation, freeze drying, or other solvent evaporation techniques, with melt-extrusion and spray-drying being preferred. The melt-extrusion process typically comprises the steps of preparing a melt which includes the active ingredient(s), the hydrophilic polymer(s) and preferably the surfactant(s), and then cooling the melt until it solidifies. “Melting” means a transition into a liquid or rubbery state in which it is possible for one component to get embedded, preferably homogeneously embedded, in the other component or components. In many cases, the polymer component(s) will melt and the other components including the active ingredient(s) and surfactant(s) will dissolve in the melt thereby forming a solution. Melting usually involves heating above the softening point of the polymer(s). The preparation of the melt can take place in a variety of ways. The mixing of the components can take place before, during or after the formation of the melt. For example, the components can be mixed first and then melted or be simultaneously mixed and melted. The melt can also be homogenized in order to disperse the active ingredient(s) efficiently. In addition, it may be convenient first to melt the polymer(s) and then to mix in and homogenize the active ingredient(s). In one example, all materials except surfactant(s) are blended and fed into an extruder, while the surfactant(s) is molten externally and pumped in during extrusion.

To start a melt-extrusion process, the active ingredient(s) (e.g., Compound 1, Compound 2 and ritonavir) can be employed in their solid forms, such as their respective crystalline forms. The active ingredient(s) can also be employed as a solution or dispersion in a suitable liquid solvent such as alcohols, aliphatic hydrocarbons, esters or, in some cases, liquid carbon dioxide. The solvent can be removed, e.g. evaporated, upon preparation of the melt.

Various additives can also be included in the melt, for example, flow regulators (e.g., colloidal silica), binders, lubricants, fillers, disintegrants, plasticizers, colorants, or stabilizers (e.g., antioxidants, light stabilizers, radical scavengers, and stabilizers against microbial attack).

The melting and/or mixing can take place in an apparatus customary for this purpose. Particularly suitable ones are extruders or kneaders. Suitable extruders include single screw extruders, intermeshing screw extruders or multiscrew extruders, preferably twin screw extruders, which can be corotating or counterrotating and, optionally, be equipped with kneading disks. It will be appreciated that the working temperatures will be determined by the kind of extruder or the kind of configuration within the extruder that is used. Part of the energy needed to melt, mix and dissolve the components in the extruder can be provided by heating elements. However, the friction and shearing of the material in the extruder may also provide a substantial amount of energy to the mixture and aid in the formation of a homogeneous melt of the components.

The melt can range from thin to pasty to viscous. Shaping of the extrudate can be conveniently carried out by a calender with two counter-rotating rollers with mutually matching depressions on their surface. The extrudate can be cooled and allowed to solidify. The extrudate can also be cut into pieces, either before (hot-cut) or after solidification (cold-cut).

The solidified extrusion product can be further milled, ground or otherwise reduced to granules. The solidified extrudate, as well as each granule produced, comprises a solid dispersion, preferably a solid solution, of the active ingredient(s) in a matrix comprised of the hydrophilic polymer(s) and the pharmaceutically acceptable surfactant(s). The extrusion product can also be blended with other active ingredient(s) and/or additive(s) before being milled or ground to granules. The granules can be further processed into suitable solid oral dosage forms.

In one example, copovidone and one or more surfactants (e.g., vitamin E TPGS in combination with propylene glycol monolaurate) are mixed and granulated, followed by the addition of aerosil and Compound 1, Compound 2 and ritonavir. The mixture is milled, and then subject to extrusion. The extrudate thus produced can be milled and sieved for further processing to make capsules or tablets. Surfactant(s) employed in this example can be added, for example, through liquid dosing during extrusion.

The approach of solvent evaporation, via spray-drying, provides the advantage of allowing for processability at lower temperatures, if needed, and allows for other modifications to the process in order to further improve powder properties. The spray-dried powder can then be formulated further, if needed, and final drug product is flexible with regards to whether capsule, tablet or any other solid dosage form is desired.

Exemplary spray-drying processes and spray-drying equipment are described in K. Masters, SPRAY DRYING HANDBOOK (Halstead Press, New York, 4^{th} ed., 1985). Non-limiting examples of spray-drying devices that are suitable for the present invention include spray dryers manufactured by Niro Inc. or GEA Process Engineering Inc., Buchi Labortechnik AG, and Spray Drying Systems, Inc. A spray-drying process generally involves breaking up a liquid mixture into small droplets and rapidly removing solvent from the droplets in a container (spray drying apparatus) where there is a strong driving force for evaporation of solvent from the droplets. Atomization techniques include, for example, two-fluid or pressure nozzles, or rotary atomizers. The strong driving force for solvent evaporation can be provided, for example, by maintaining the partial pressure of solvent in the spray drying apparatus well below the vapor pressure of the solvent at the temperatures of the drying droplets. This may be accomplished by either (1) maintaining the pressure in the spray drying apparatus at a partial vacuum; (2) mixing the liquid droplets with a warm drying gas (e.g., heated nitrogen); or (3) both.

The temperature and flow rate of the drying gas, as well as the spray dryer design, can be selected so that the droplets are dry enough by the time they reach the wall of the apparatus. This help to ensure that the dried droplets are essentially solid and can form a fine powder and do not stick to the apparatus wall. The spray-dried product can be collected by removing the material manually, pneumatically, mechanically or by other suitable means. The actual length of time to achieve the preferred level of dryness depends on the size of the droplets, the formulation, and spray dryer operation. Following the solidification, the solid powder may stay in the spray drying chamber for additional time (e.g., 5-60 seconds) to further evaporate solvent from the solid powder. The final solvent content in the solid dispersion as it exits the dryer is preferably at a sufficiently low level so as to improve the stability of the final product. For instance, the residual solvent content of the spray-dried powder can be less than 2% by weight. Highly preferably, the residual solvent content is within the limits set forth in the International Conference on Harmonization (ICH) Guidelines. In addition, it may be useful to subject the spray-dried composition to further drying to lower the residual solvent to even lower levels. Methods to further lower solvent levels include, but are not limited to, fluid bed drying, infra-red drying, tumble drying, vacuum drying, and combinations of these and other processes.

Like the solid extrudate described above, the spray dried product contains a solid dispersion, preferably a solid solution, of the active ingredient(s) in a matrix comprised of the hydrophilic polymer(s) and the pharmaceutically acceptable surfactant(s).

Before feeding into a spray dryer, the active ingredient(s) (e.g., Compound 1, Compound 2 and ritonavir), the hydrophilic polymer(s), as well as other excipients such as the pharmaceutically acceptable surfactant(s), can be dissolved in a solvent. Suitable solvents include, but are not limited to, alkanols (e.g., methanol, ethanol, 1-propanol, 2-propanol or mixtures thereof), acetone, acetone/water, alkanol/water mixtures (e.g., ethanol/water mixtures), or combinations thereof. The solution can also be preheated before being fed into the spray dryer.

The solid dispersion produced by melt-extrusion, spray-drying or other techniques can be prepared into any suitable solid oral dosage forms. In one embodiment, the solid dispersion prepared by melt-extrusion, spray-drying or other techniques (e.g., the extrudate or the spray-dried powder) can be compressed into tablets. The solid dispersion can be either directly compressed, or milled or ground to granules or powders before compression. Compression can be done in a tablet press, such as in a steel die between two moving punches.

At least one additive selected from flow regulators, binders, lubricants, fillers, disintegrants, or plasticizers may be used in compressing the solid dispersion. These additives can be mixed with ground or milled solid dispersion before compacting. Disintegrants promote a rapid disintegration of the compact in the stomach and keeps the liberated granules separate from one another. Non-limiting examples of suitable disintegrants are cross-linked polymers such as cross-linked polyvinyl pyrrolidone, cross-linked sodium carboxymethylcellulose or sodium croscarmellose. Non-limiting examples of suitable fillers (also referred to as bulking agents) are lactose monohydrate, calcium hydrogenphosphate, microcrystalline cellulose (e.g., Avicell), silicates, in particular silicium dioxide, magnesium oxide, talc, potato or corn starch, isomalt, or polyvinyl alcohol. Non-limiting examples of suitable flow regulators include highly dispersed silica (e.g., colloidal silica such as Aerosil), and animal or vegetable fats or waxes. Non-limiting examples of suitable lubricants include polyethylene glycol (e.g., having a molecular weight of from 1000 to 6000), magnesium and calcium stearates, sodium stearyl fumarate, and the like.

Various other additives or ingredients may also be used in preparing a solid composition of the present invention, for example dyes such as azo dyes, organic or inorganic pigments such as aluminium oxide or titanium dioxide, or dyes of natural origin; stabilizers such as antioxidants, light stabilizers, radical scavengers, stabilizers against microbial attack; or other active pharmaceutical ingredients.

In order to facilitate the intake of a solid dosage form, it is advantageous to give the dosage form an appropriate shape. Large tablets that can be swallowed comfortably are therefore preferably elongated rather than round in shape.

A film coat on the tablet further contributes to the ease with which it can be swallowed. A film coat also improves taste and provides an elegant appearance. The film-coat usually includes a polymeric film-forming material such as hydroxypropyl methylcellulose, hydroxypropylcellulose, and acrylate or methacrylate copolymers. Besides a film-forming polymer, the film-coat may further comprise a plasticizer, e.g. polyethylene glycol, a surfactant, e.g. polysorbates, and optionally a pigment, e.g. titanium dioxide or iron oxides. The film-coating may also comprise talc as anti-adhesive. Preferably, the film coat accounts for less than 5% by weight of a pharmaceutical composition of the present invention.

It should be understood that the above-described embodiments and the following examples are given by way of illustration, not limitation. Various changes and modifications within the scope of the present invention will become apparent to those skilled in the art from the present description.

### EXAMPLE 1

Compound 1, Compound 2 and ritonavir were extruded using melt-extrusion. 31 g of copovidone, 3.5 g of vitamin E TPGS and 1.0 g of propylene glycol monolaurate were granulated for 2 minutes in a mill. After two hours, the mixture was blended with 5.09 g of ritonavir, 1.27 g of Compound 2, 7.64 g of Compound 1 and 0.5 g colloidal silica for 2 minutes in a blender and subsequently sieved over a 1.0 mm mesh sieve. The mixture was extruded on a Haake/Thermo Fisher extruder at 165° C. at a screw speed of 30 rpm. The extrudate thus prepared contained 15% Compound 1, 10% ritonavir, 2.5% Compound 2, 62% copovidone, 1% colloidal silica (areosil 200), 7% vitamin E TPGS, and 2% propylene glycol monolaurate.

The extrudate was then milled, and blended with sodium stearyl fumarate in a blender for 3 minutes, sieved and blended again in the blender for another 3 min. The blend was compressed to tablets on a tablet press.

The tablet was tested in a pH dilution drug release model (pH change from 0.1 N HCl to pH 6.8 phosphate buffer) and showed excellent drug release profile. Over 60% of the drugs in the tablet were released within 60 minutes.

The tablet was also tested for stability. After 4-week storage at 40° C. and 75% relative humidity, no evidence for residual or recrystallized drug substance was found. The degradation of each drug substance after melt extrusion was within the specifications.

What is claimed is:

1. A solid pharmaceutical composition comprising: 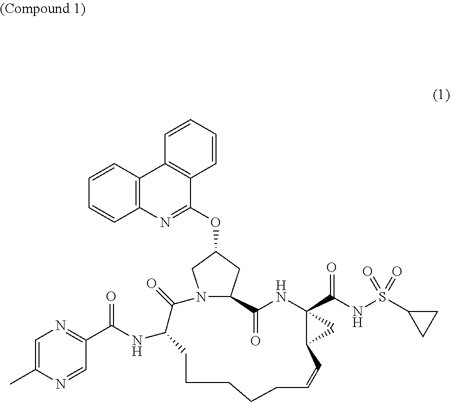 [SMILES: Cc1cnc(C(=O)N[C@H]2CCCCC/C=C\[C@@H]3C[C@@]3(C(=O)NS(=O)(=O)C3CC3)NC(=O)[C@@H]3C[C@@H](Oc4nc5ccccc5c5ccccc45)CN3C2=O)cn1]
or a pharmaceutically acceptable salt thereof, 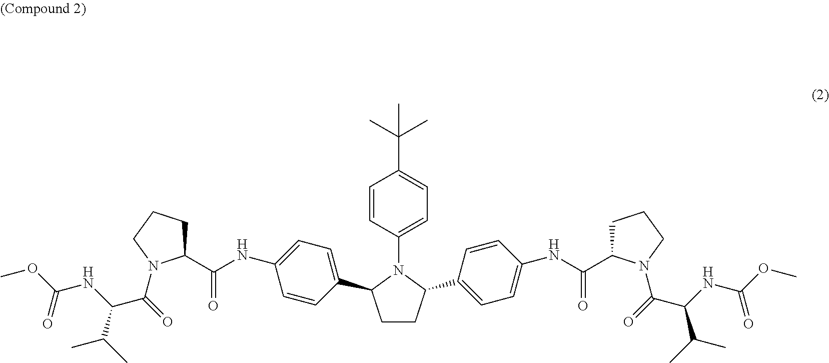
or a pharmaceutically acceptable salt thereof,
(3) ritonavir or a pharmaceutically acceptable salt thereof,
(4) a pharmaceutically acceptable hydrophilic polymer, and
(5) a pharmaceutically acceptable surfactant,
all of which are co-formulated in amorphous solid dispersion.

2. The pharmaceutical composition of claim 1, wherein said polymer is copovidone, and said surfactant is vitamin E TPGS.

3. The pharmaceutical composition of claim 2, wherein said amorphous solid dispersion further comprises propylene glycol monolaurate.

4. The pharmaceutical composition of claim 1, comprising 75 mg Compound 1, 12.5 mg Compound 2, and 50 mg ritonavir, all of which are co-formulated with said polymer and said surfactant in amorphous solid dispersion.

5. The pharmaceutical composition of claim 1, comprising 75 mg Compound 1, 12.5 mg Compound 2, and 50 mg ritonavir, all of which are co-formulated with said polymer and said surfactant in said amorphous solid dispersion, wherein said polymer in said amorphous solid dispersion ranges from 50% to 70% by weight relative to the total weight of said amorphous solid dispersion, and said surfactant in said amorphous solid dispersion ranges from 5% to 15% by weight relative to the total weight of said amorphous solid dispersion.

6. The pharmaceutical composition of claim 1, comprising 75 mg Compound 1, 12.5 mg Compound 2, and 50 mg ritonavir, all of which are co-formulated with said polymer and said surfactant in said amorphous solid dispersion, wherein said polymer in said amorphous solid dispersion ranges from 55% to 65% by weight relative to the total weight of said amorphous solid dispersion, and said surfactant in said amorphous solid dispersion ranges from 5% to 10% by weight relative to the total weight of said amorphous solid dispersion.

7. The pharmaceutical composition of claim 1, comprising 75 mg Compound 1, 12.5 mg Compound 2, and 50 mg ritonavir, all of which are co-formulated with said polymer and said surfactant in said amorphous solid dispersion, wherein said polymer in said amorphous solid dispersion ranges from 60% to 65% by weight relative to the total weight of said amorphous solid dispersion, and said surfactant in said amorphous solid dispersion ranges from 5% to 10% by weight relative to the total weight of said amorphous solid dispersion.

8. The pharmaceutical composition of claim 4, wherein said polymer is copovidone, and said surfactant is vitamin E TPGS.

9. The pharmaceutical composition of claim 8, further comprising propylene glycol monolaurate which is co-formulated with said Compound 1, Compound 2, ritonavir, copovidone and vitamin E TPGS in said amorphous solid dispersion.

10. The pharmaceutical composition of claim 9, wherein said propylene glycol monolaurate in said amorphous solid dispersion ranges from 1% to 5% by weight relative to the total weight of said amorphous solid dispersion.

11. The pharmaceutical composition of claim 1, comprising a compressed core which includes 75 mg Compound 1, 12.5 mg Compound 2, and 50 mg ritonavir, all of which are co-formulated with said polymer and said surfactant in said amorphous solid dispersion, wherein the total weight of said compressed core is no more than 700 mg.

12. The pharmaceutical composition of claim 1, comprising a compressed core which includes 75 mg Compound 1, 12.5 mg Compound 2, and 50 mg ritonavir, all of which are co-formulated with said polymer and said surfactant in said amorphous solid dispersion, wherein the total weight of said compressed core is no more than 600 mg.

13. The pharmaceutical composition of claim 1, comprising a compressed core which includes 75 mg Compound 1, 12.5 mg Compound 2, and 50 mg ritonavir, all of which are co-formulated with said polymer and said surfactant in said amorphous solid dispersion, wherein the total weight of said compressed core is no more than 500 mg.

14. The pharmaceutical composition of claim 11, wherein said polymer is copovidone, and said surfactant is vitamin E TPGS.

15. The pharmaceutical composition of claim 14, wherein propylene glycol monolaurate is co-formulated with said Compound 1, Compound 2, ritonavir, copovidone and vitamin E TPGS in said amorphous solid dispersion.

16. The pharmaceutical composition of claim 11, wherein said polymer in said amorphous solid dispersion ranges from 50% to 70% by weight relative to the total weight of said compressed core, and said surfactant in said amorphous solid dispersion ranges from 5% to 15% by weight relative to the total weight of said compressed core.

17. The pharmaceutical composition of claim 11, wherein said polymer in said amorphous solid dispersion ranges from 55% to 65% by weight relative to the total weight of said compressed core, and said surfactant in said amorphous solid dispersion ranges from 5% to 10% by weight relative to the total weight of said compressed core.

18. The pharmaceutical composition of claim 11, wherein said polymer in said amorphous solid dispersion ranges from 60% to 65% by weight relative to the total weight of said compressed core, and said surfactant in said amorphous solid dispersion ranges from 5% to 10% by weight relative to the total weight of said compressed core.

19. The pharmaceutical composition of claim 16, wherein propylene glycol monolaurate is co-formulated with said Compound 1, Compound 2, ritonavir, polymer and surfactant in said amorphous solid dispersion, and said propylene glycol monolaurate in said amorphous solid dispersion ranges from 1% to 5% by weight relative to the total weight of said compressed core.

20. A process of making a pharmaceutical composition of claim 1, comprising:
(1) preparing a melt comprising said Compound 1 (or a pharmaceutically acceptable salt thereof), said Compound 2 (or a pharmaceutically acceptable salt thereof), said ritonavir (or a pharmaceutically acceptable salt thereof), said pharmaceutically acceptable polymer, and said pharmaceutically acceptable surfactant; and
(2) solidifying said melt.
